(19) United States
(12) Patent Application Publication
(10) Pub. No.: US 2026/0097186 A1
(43) Pub. Date: Apr. 9, 2026

(54) GUIDE ELEMENT FOR INTRAVASCULAR ACCESS DEVICE

(71) Applicant: VenoCare, Inc., Doral, FL (US)

(72) Inventors: Raul LEYTE-VIDAL, Doral, FL (US); Ryan PATTERSON, Kaysville, UT (US); Amir BELSON, Savyon (IL)

(21) Appl. No.: 19/113,880

(22) PCT Filed: Sep. 20, 2023
(86) PCT No.: PCT/US2023/074699

§ 371 Date: Mar. 20, 2025

Related U.S. Application Data
(60) Provisional application No. 63/408,419, filed on Sep. 20, 2022.

(51) Int. Cl. A61M 25/06; A61M 25/09

(52) U.S. Cl. CPC A61M 25/0606; A61M 25/09; A61M 2025/09116

(57) ABSTRACT

A guide element for an intravascular access device may include an intravascular access device having a needle inside of a catheter lumen and a working space between an exterior surface of the needle and an interior surface of the catheter, the working space configured to receive one or more tools therethrough, and a guide element having a distal end and a proximal end separated by one or more annular elements, the guide element configured to be distally advanced from the intravascular access device inside of a blood vessel, wherein the one or more annular elements is configured to control at least a distal end of the guide element.

### CLAIM OF PRIORITY

This patent application claims priority to U.S. Provisional Ser. No. 63/408,419, filed Sep. 20, 2022, titled “GUIDE ELEMENT FOR INTRAVASCULAR ACCESS DEVICE”, which is incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Intravenous access, such as venipuncture, is a necessary element for several medical procedures. Venipuncture refers generally to the process of obtaining intravenous access for any one of a variety of purposes, including intravenous infusion, therapy, blood sampling, and the like. In the hospital, for example, venipuncture is commonly used to place a small intravenous catheter for delivering intravenous fluids, drug delivery, blood sampling and the like.

While venipuncture and other forms of vascular access in relatively healthy patients can be a simple matter, such access is often needed in patients who are not healthy and may have small, tortuous, collapsed, fragile, and/or difficult to locate arteries and/or veins. In such patients, venipuncture and other forms of vascular access can be very challenging, particularly to less experienced phlebotomists, paramedics, nurses, and other health care practitioners.

In addition to difficult access, many vascular catheter placement systems can result in accidental punctures and/or accidental needle contamination during or after placement of the intravascular catheter. Still further, some conventional catheter placement devices employ relatively complex deployment handle movements that lead to increases both cost and complexity. Additionally, conventional handle placement and movements can obscure the presence and status of the needle and guide structure or guide element components of the tool, thus making use of the insertion tool less intuitive.

For these reasons, it would be desirable to provide improved methods, systems, and tools for deploying intravascular catheters using needles and guide structures. It would be particularly desirable to provide simplified deployment systems and assemblies having fewer components and, even more desirably, to provide components which are clearly visible to the user and configured to be utilized and manipulated in a straightforward, intuitive manner. At least some of these objectives will be met by the various embodiments that follow.

### SUMMARY OF THE DISCLOSURE

Described herein are guide elements, intravascular access devices and methods of using the same.

In general, a guide element for an intravascular access device can comprise a catheter body having a distal end, a proximal end and a lumen extending therethrough, an intravascular access device having a needle inside of the catheter lumen and a working space between an exterior surface of the needle and an interior surface of the catheter, the working space can be configured to receive one or more tools therethrough, a guide element within the working space wherein a portion of the catheter body adjacent to the guide element is displaced in response to the distal movement of the guide element along the needle within the working space.

This and other examples described herein may also comprise any of the following: The portion of the catheter body can be displaced by moving a flap formed on the catheter body in response to the guide element. The portion of the catheter body can be displaced by the guide element having a different durometer than the rest of the catheter body. The portion of the catheter body having a different durometer that may be displaced by movement of the guide element can be an annular portion at a distal most portion of the catheter body. The portion of the catheter body having a different durometer that may be displaced by movement of the guide element can be adjacent to the working space or in a semicircular portion that corresponds to a flat portion of the needle. The catheter body may have a first portion formed from a material of a first durometer and a second portion formed of a material of a second durometer wherein the first portion may be adjacent to the working space and the first durometer can be selected such that in use as a distal end of the guide element is advanced against the first portion, the first portion deflects or deforms in response.

In general, a guide element for use with an intravascular access device may comprise an intravascular access device having a needle inside of a catheter lumen and a working space between an exterior surface of the needle and an interior surface of the catheter, the working space can be configured to receive one or more tools therethrough, a guide element may have a distal end and a proximal end separated by one or more annular elements, the guide element can be configured to be distally advanced from the intravascular access device inside of a blood vessel, wherein the one or more annulare elements may be configured to control at least a distal end of the guide element.

In some examples, the intravascular guide element may also have at least two annular elements, wherein a first annular element can be configured to control a direction of travel of the guide element distal end. The distal end of the guide element can be an atraumatic tip. The distal end of the guide element can be a formed distal tip. The guide elements can be comprised of a plurality of segments. The intravascular guide element may further comprise at least four annular elements. The needle may have at least one flat surface, and wherein the working space is defined by an interior arc of the catheter interior surface and the needle flat surface. The distal tip may further comprise one or more deployment slits aligned with the working space. The catheter distal tip may further comprise one or more conforming segments, wherein a distal tip of the guide element can be configured to engage the one or more confirming segments.

In general, an intravascular access device may include a handle having a proximal end and a distal end, a slot extending from the proximal end to the distal end. A catheter may have a proximal catheter hub and a distal catheter lumen, the proximal catheter hub can be releasably engaged to the distal end of the handle. An access needle extending proximally from within a needle carrier through the catheter lumen, the access needle having at least one flat surface extending longitudinally along a perimeter of the access needle, and a tissue-penetrating tip may extend distally beyond the catheter lumen. Also included may be a working space within the catheter along a length of the access needle at least one flat, convex, or concave surface or, wherein the working space is configured to facilitate passage of one or more tool therethrough from a proximal end of the intravascular access device distally into a vessel. A slide can extend through the slot, the slide may be in communication with a proximal end of the guide element so that distal advancement of the slide advances a distal tip portion of the guide element from a position within a guide element cutout distally along the at least one flat surface of the access needle.

In some examples, the at least one flat surface of the access needle extends longitudinally down an exterior surface of the access needle. The at least one tool comprises a guide element. The working space may be defined by the needle flat surface and an interior surface of the catheter, wherein the working space is selectively accessible from a proximal end of the intravascular access device. The working space can be configured to selectively open at the catheter distal end and wherein the working space is configured to guide a tool or tool segment sliding therethrough. The intravascular access device may further include an access needle lumen, wherein the guide element may comprise a plurality of annular elements between a distal end and proximal end, wherein one or more of the annular elements may extend through the access needle lumen. The intravascular access device may further include a hemostasis valve having longitudinal channels around a perimeter of the homeostasis valve, wherein the hemostasis valve can be disposed within the proximal catheter hub. The access needle comprises a plurality of flat surfaces, wherein each of the plurality of flat surfaces can be associated with a separate working space within the catheter lumen.

In some examples, the intravascular access device may further include an actuation button coupled to the needle carrier, and an actuation element exerting a force on the needle carrier towards the proximal end of the handle, wherein when the actuation button is depressed the actuation element displaces the needle carrier and access needle toward the proximal end of the handle. The access needle is retracted proximally towards the handle, and wherein the guide element may be configured to remain in a distally advanced position. The intravenous access device may further include a spool of guide element in communication with the proximal end of the intravascular device, wherein a length of the guide element can be contained within the spool. The guide element may be made entirely or partially of a metallic material, polymeric material, or a combination thereof. The guide element may comprise a plurality of segments, wherein one or more of the plurality of segments may comprise a different material.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is an illustration of an example of a guide wire element, as described herein, used with an intravascular access device.

FIG. 2 illustrates an atraumatic guide wire element distal end used with an intravascular access device according to examples described herein.

FIG. 3A and FIG. 3B illustrate details of an arrangement of a guide wire element associated with an intravascular access device according to examples described herein

FIG. 4 shows an example of deployment of a guide element advancing outward from an intravascular access device according to examples described herein.

FIG. 5A and FIG. 5B are illustrations of distal end detail examples of a guide element described herein.

FIG. 6A, FIG. 6B and FIG. 6C are cross sectional views of an intravascular access device and examples of a transition from a retracted configuration to a deployed configuration according to examples described herein.

FIG. 7A and FIG. 7B show truncated cross-sectional view of an intravascular access device showing a transition of the guide element from a retracted position to a deployed position according to examples described herein.

FIG. 8A to FIG. 8F shows illustrative examples of the guide element configuration along a length of the guide element including the distal end according to examples described herein.

FIG. 9A to FIG. 9D are illustrations of a multi-filament guide element including different configurations according to examples described herein.

FIG. 10A and FIG. 10B illustrate distal end configurations of an intravascular access device according to examples described here.

FIG. 11A to FIG. 11C illustrate examples of distal end configurations of an intravascular access device according to examples described here.

FIG. 12A and FIG. 12B illustrate examples of distal end configurations of an intravascular access device according to examples described here.

FIG. 13A and FIG. 13B are cross sectional views illustrating examples of distal end configurations of an intravascular access device according to examples described here.

FIG. 14A to FIG. 14C are cross section views that illustrate examples of guide element elongate body configurations as described herein.

FIG. 15A to FIG. 15C illustrate examples of a catheter distal portion with details of a guide element distal tip, as described herein.

FIG. 16 shows an example of a guide element in use with an intravascular access device.

### DETAILED DESCRIPTION

An intravascular access device may include a needle at least partially disposed within a catheter. The needle can be configured to puncture and traverse one or more layers of tissue until access to a blood vessel is achieved. The catheter can advance through one or more layers of tissue as the needle is advanced accordingly. Once inside of a vessel, it may be desirable for the catheter to advance beyond the needle into the vessel. A guide element generally positioned between an exterior surface of the needle and an interior surface of the catheter may be selectively manipulated (e.g., advanced) ahead of the catheter distal tip to aid in guiding and placement of the catheter in the vessel distal to the point of insertion. The guide element may have one or more features providing increased function, control, and safety.

An intravascular access device, as described herein, may comprise a working space or working channel created by a needle having a semi-circular cross-sectional geometry. Current intravascular access devices provide for concentric circular cross-sectional geometry between the needle and catheter (e.g., a tubular needle within a tubular catheter). The intravascular access devices described herein provide a needle with a semi-circular cross-sectional geometry. For example, a needle having a circumference comprising an arc and a generally flat segment. In some examples, the arc portion of the needle circumference can be less than 360 degrees, less than 350 degrees, less than 340 degrees, less than 330 degrees, less than 320 degrees, less than 310 degrees, less than 300 degrees, less than 270 degrees, less than 180 degrees, or more. In some examples, the flat surface of the needle defines a discontinuous circular circumference (e.g., a circle with a segment removed). In some examples, the flat segment on the perimeter of the needle may relate to a missing minor segment from an otherwise complete circumference.

Dimensions of the working space may be an area of the missing minor segment of the needle. For examples, considering a cross-section of the needle and catheter at any point along their length, an area of the working space may be calculated as the difference between the area of the needle and the area of the circular cross-section of the catheter. For example, at any cross section of the catheter an area may be calculated as πr², r being a radius of the catheter circular cross-section; and an area of the needle may be calculated as πr²-the area of the missing minor segment. Accordingly, the working space may be the difference between the area of the interior of the catheter and the area of the needle.

The working space may be configured to accommodate one or more tools (e.g., guide elements). In some examples, the working space between the exterior of the needle and the interior surface of the catheter may be configured to accommodate a guide element, as described herein. In some examples, the working space may be configured to accommodate procedurally related tools. For example, a tool may be selectively passed into and/or through the working space from a proximal end of the intravascular access device to a distal end or distal tip of the intravascular access device (e.g., a distal tip of the catheter).

The working space may be configured to accommodate the administration of one or more therapeutics during an intravascular access procedure. For example, the distal tip of the needle may first penetrate and traverse biological tissue until entering a vessel. The catheter of the intravascular access device may traverse the biological tissue with the needle until the distal tip of the catheter is within the vessel. The operator may then introduce a tool from a proximal end of the intravascular access device accessible outside of the patient's body and advance the tool through the intravascular access device such that the tool passes or slides within the working space between the needle exterior surface and the interior surface of the catheter until it is functionally deployed within the vessel as allowed by the positioning of the catheter distal tip. The operator may engage a proximal end of the tool and/or a proximal end of the intravascular access device to control the tool within the blood vessel. After a procedure is complete, the operator may retract the tool through the working space allowing the intravascular access device to remain in position within the vessel.

According to any example described herein, it may be useful to guide an intravascular access device into and/or through a patient's vasculature. For example, the intravascular access device may first penetrate and traverse biological tissue until entering a vessel. The catheter of the intravascular access device may need to be advanced beyond the initial placement into the vessel facilitated by the needle distal tip. Accordingly, a guide element may be deployed in advance of the catheter deployment to aid in the routing and positioning of the catheter through distal areas of the vasculature. A guide element, as described herein may include a distal end and a proximal end separated by a length of guide element body configured to pass through an intravascular access device having a working space between the needle and an interior of the catheter.

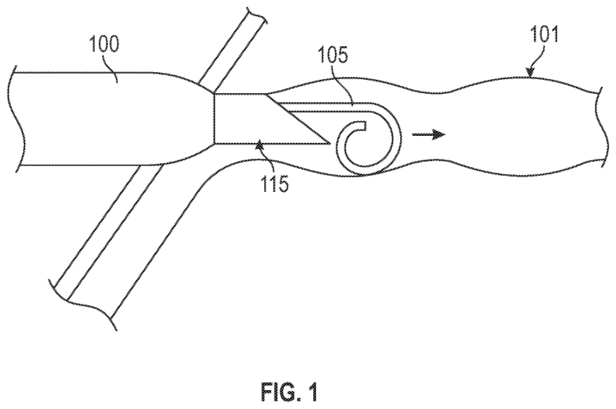

FIG. 1 illustrates an example of an intravascular access device 100, as described herein, having been interested into a vessel 101 of a patient. An example of a guide element feature is illustrated where the distal end of the guide element 105 is curled and forms an atraumatic distal end. The advantage of an atraumatic tip or distal end of the guide element, as described herein, is to prevent unintended perforation of the vessel by the guide element or other adverse events such as snagging of the guide element distal end by the patient's vascular anatomy. When the needle 115 has been inserted into the vessel 101, the guide element distal end 105 can be advanced into the vessel and may curl according to a shape memory of the guide element material (e.g., nitinol).

In some examples a guide element, as described herein, may have a distal end configured to engage an interior of a vessel. For example, the distal end may include the distal tip (e.g., the distal terminal end) and a segment of the guide element body. As illustrated in FIG. 1, the curled atraumatic distal end of the guide element may be configured to advance through a vessel and maintain a geometry as it is advanced through the vessel. In some examples, the distal end of the guide element may apply a force against an interior of the vessel to open or otherwise facilitate passage of the catheter through a collapsed or partially collapsed vessel.

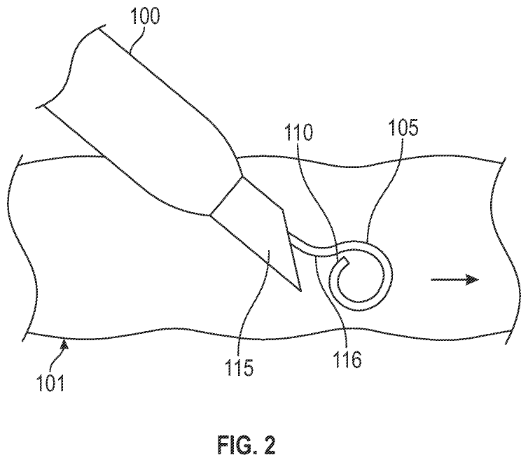

FIG. 2 illustrates an example of a guide element 105, as described herein. Similar to FIG. 1, the guide element distal end 110 is curled forming an atraumatic tip. In some examples, the guide element is generally linear at the distal end in a retracted or ready configuration and may automatically confirm to an atraumatic distal end as the guide element 105 is advanced distally from the intravascular access device 100. In FIG. 2, the guide element is shown as advancing from within the needle 115. The needle 115 may have an aperture extending therethrough from the needle distal tip 116 to a proximal end of the need (not shown). The guide element may be configured to pass or be advanced through the needle aperture into the vasculature of the patient.

The distal end of the guide element may be flexible at one or more segments including the distal end and/or one or more segments of the guide element body. For example, FIG. 2 illustrates the guide element having been advanced beyond the needle distal tip and, after contacting an interior surface of the vessel, the guide element bends against the interior vessel surface continuing into the vessel as facilitated by the curved distal end.

In some examples, the guide element may be initially positioned in the intravascular device such that the guide element slides through the needle aperture and distally further into the vessel beyond the needle tip. In such a configuration, the needle may be retractable towards a proximal end of the intravascular access device to allow the guide element to remain in position, or advanceable into the vasculature without requiring the needle to remain. Such a configuration may reduce the amount of traumatic impact on the vessel as the needle may be entirely removed from the vessel and/or from the patient's anatomy while the guide element remains effective to guide the catheter as it is advanced into the vessel along the guide element path.

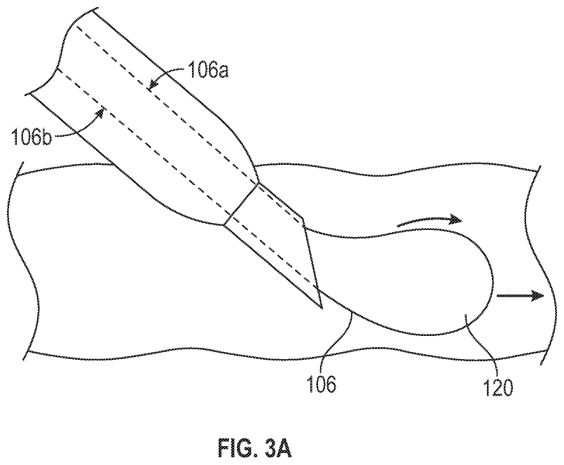

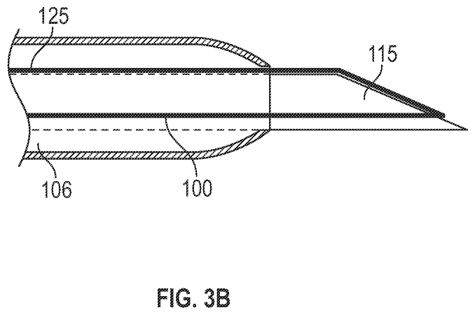

The guide element may initially be disposed in one or more retracted or ready configurations throughout the intravascular access device. In some examples, the guide element may be generally linear from the distal end to a proximal end and may be configured to slide through the intravascular access device through the working space between the needle exterior surface and the catheter interior surface. In some examples, the guide element may be configured to slide through the intravascular access device via the needle aperture. In some examples, the guide element may be configured to advance distally from the distal end of the intravascular access device through the needle aperture while looping proximally through the working spare of the intravascular access device, as described herein. In some examples, the guide element may loop into the same area or space from which it was advanced. For example, in FIG. 3A, a guide element distal loop 120 is illustrated wherein the guide element 106 is advanced from within the needle aperture. Dotted lines are included to illustrate an example of the distal end doubling back through the needle aperture towards a proximal end of the intravascular access device. In some examples, the guide element may be a single length of one or more materials and may be advanced into a vessel through an intravascular access device led by a point of the guide element between the distal and proximal ends. In some examples, the guide element may comprise a loop of material that may be advanced through the intravascular access device. In the example illustrated in FIG. 3A, the guide element 106 may have a first side 106a and second side 106b of the elongate body. Accordingly, either side 106a or 106b may be advanced to provide the loop 120 advancing in the vessel. For example, the loop 120 may roll distally outward as one side or both sides of the guide element 106 are advanced distally. FIG. 3B is a closeup view of the distal end of the intravascular access device illustrating an example of the guide element 106. Neither the proximal end or the distal end of the guide element are shown. However, the guide element can be seen positioned at least partially inside the needle aperture and partially within the working space of the intravascular access device 100. In this configuration, the guide element may be selectively advanced such that the operator may advance the guide element distally from the intravascular access device through the needle aperture, or through the working space, or some combination of both. For example, the intravascular access device may be positioned within the vessel and the operator may first attempt to advance the guide element distally through the working space, then may advance the guide element through the needle aperture to direct the advancement of the guide element through the vasculature, then the operator may advance both the working space side of the guide element and the needle aperture side of the guide element simultaneously. In some examples, advancing both sides (e.g., both ends) of the guide element distally may create a loop or may adjust the size of a loop created as the guide element is advanced distally from the intravascular access device. For example, if both sides of the guide element are advanced, the size of the loop may increase as the guide element body at the loop may bias the guide element into an open position, thereby increasing the loop size.

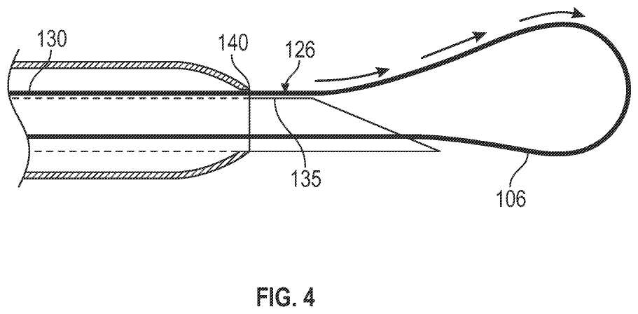

As described above, FIG. 4 illustrates a transition from a retracted position to an advancing position wherein a first side 126 of the guide element 106 is being advanced shows the element advanced through the working space 130 while the needle 115 remains stationary. Of note, the working space 130 is visible and the intravascular access device 100 is oriented to show the needle flat surface 135 partially extending outwards from the catheter distal end 140. It can be appreciated that the flat surface of the needle functions as a platform for the portion of the guide element 106 positioned within the working space 130. Although not expressly shown, the interior of the catheter may be a continuous circular interior surface to form the working space 130 with the flat needle surface 135.

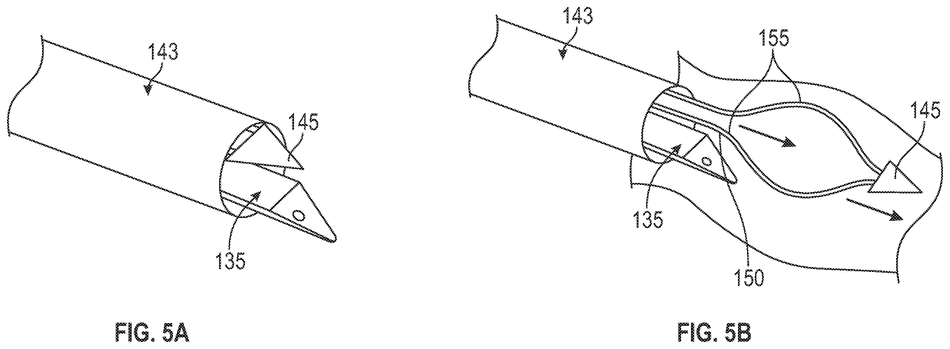

FIGS. 5A and 5B illustrate examples of a guide element, as described herein having a formed distal tip 145. The guide element distal tip may be defined by a portion of the guide element (e.g., the distal end or distal end segment) or by a formed distal end. Formed distal ends of a guide element described herein may include distal ends that are welded or otherwise attached to a distal tip of the guide element. In FIG. 5A, the formed distal end 145 may be molded with the guide element during initial manufacture or may be affixed to the guide element at some time after. In some examples, the formed distal end may be a modified distal tip such that the distal tip of the guide element is modified to a shape or configuration. The example shown in FIG. 5A has the formed distal tip 145 essentially completing the needle distal end geometry to a cylinder or tubular shape that is tapered from near the distal end of the needle (e.g., near the needle tip) to the distal end of the catheter 143. In this configuration, there is a reduced impact of the distal end of the intravascular access device as it penetrates and traverses the biological tissue and vessels of a patient's anatomy, lowering the penetration force. Shown in FIG. 5B, a similar formed distal tip 145 of the guide element 150 highlights a distal end of the guide element 150 having more than one annular element 155 (e.g., body segment) extending proximally therefrom. In particular, FIG. 5B illustrates a guide element having two annular elements 155 comprising the guide element body. As described herein, guide elements having a body comprised of a plurality of annular elements increases the function of the guide element. For example, as the guide element 150 is advanced distally into the vasculature, both annular elements 155 may be advanced simultaneously and may maintain a generally parallel guide element path, relative to one another. In some examples, fewer than all of the annular elements 155 may be advanced promoting selective directional control of the guide element 150 (e.g., the guide element distal tip). For example, if the operator advanced both of the annular elements simultaneously, the guide element may proceed distally into the vasculature. If the operator advances fewer than all of the annular elements 155 at one time, the distal end of the guide element may turn or curve in a direction. In some examples, the guide element 150 may turn or curve in a direction opposite of the advance annular element. In some examples, the operator may selectively retract fewer than all of the guide wire annular elements to steer or turn the distal end of the guide element as it is advanced through the vasculature. In some examples, the distal segment of the guide element is molded, welded, shaped, or configured for a particular function.

Guide elements, described herein may comprise one or more sections, segments, portions, or regions having different materials and/or material characteristics. For example, a guide element described herein may comprise an elongate member extending from a proximal end to a distal end or region where a conforming tip may be coupled to the distal end of the elongate member. For example, a guide element described herein may have a wire (e.g., elongate/annular member) that extends from the proximal end to a distal end where a plastic, polymer, and/or other acceptable molded material is coupled thereto. In some examples, the distal region may be molded, welded, adhered, or otherwise affixed to the distal end or region of the guide element body. In some examples, the conforming tip (e.g., distal region/end) of the guide element may be manufactured onto the elongate member body of the guide element. In some examples, the elongate member body of the guide element may have one or more features (e.g., tabs, prongs, groves, grips, etc.) configured to retain the conforming tip coupled thereto. In some examples, the elongate body of the guide element may be a wire or similar element having appropriate column strength to advance or otherwise control the guide element during use (e.g., advancement into or through vasculature).

FIG. 5B also shows an example of the increased function of a guide element, as described herein. Each of the annular elements 155 bow or bend away from each other in a spring biased fashion forming a bubble or opening that may expand as the guide element is advanced further outward from the intravascular access device. The expansion of these annular elements in this manner may increase or otherwise augment a vessel opening to the benefit of the patient and/or operator during a procedure. In some examples, the formed distal tip 145 may refer to a nose core or core segment including the distal ends of each annular element 155 within or affixed to a nose or distal tip of the guide element 150.

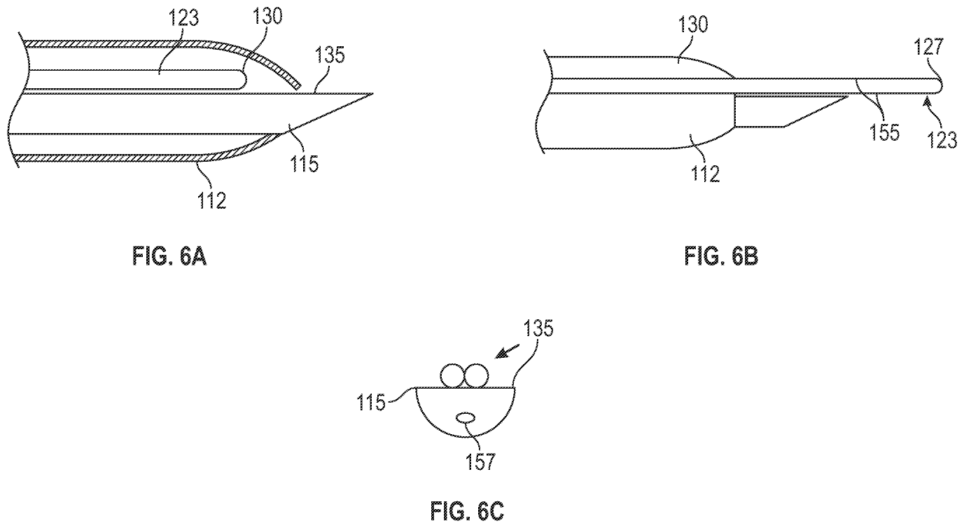

As described herein, a guide element may include an atraumatic tip. FIGS. 6A and 6B illustrate examples of a rounded or looped tip 127 of the guide element 123 formed at the distal end or distal segment such that the tip can be configured to protect the guide element upon vessel insertion by having a catheter tip compliant and allowing the element to emerge out of the distal tip of the catheter after vessel insertion. The catheter 112 is shown substantially transparent to highlight the position of the guide element in the working space. Accordingly, the annular elements 155 of the guide element 123 are configured to be advanced or retracted slidingly through the working space 130 between the flat surface of the needle and the interior of the catheter. Where FIG. 6A illustrates and example of a guide element in an extended or advanced configuration beyond the distal end of the intravascular access device 100, FIG. 6B illustrates the guide element 123 in a retracted configuration such that the entire distal end of the guide element is retracted and positioned within the catheter 112. Additionally, the distal end of the catheter 112 is shown as contacting the exterior surface of the needle, including contacting the exterior needle flat surface, which can be seen extending beyond the distal end of the catheter 112. Added in FIG. 6C is an exploded view of a cross section of the needle 115 and the annular elements 155 of the guide element 123 without the catheter. In particular, the needle flat surface extends across a section of the needle perimeter while allowing for the needle aperture 157.

In some embodiments, the guide element can be retained in a ready configuration or position within the distal end of the intravascular access. For example, before, during, and/or after the intravascular access device is inserted into the patient's vasculature, the guide element may continue to be retrained in the ready configuration within the catheter interior. The needle may slide distally and/or proximally without disrupting the position of the guide element in this configuration. Similarly, the catheter may also be advanced or retracted along the needle with the guide elements continuously retained in the ready configuration. For example, the guide element may be retained inside the working space, as described herein, while the need is initially advanced or extended distally in preparation of tissue penetration, and the guide element may be retained within the working space by the tapered catheter distal end.

The guide element structures may be configured or have a geometry to compliment the working space, the needle flat surface, the interior surface of the catheter, the needle aperture, the needle distal end, the needle distal tip, procedural parameters, the guide element function, guide element steerability, etc. For example, the guide element may be geometrically configured to conform to the needle exterior and the needle exterior may be curved such that the guide element cross-sectional geometry is concave to compliment the curved exterior of the needle. In other examples, the needle flat surface may be compliments by one or more of the guide element annular elements having a correspond flat surface. In some examples, the geometry of each of the annular elements may complement each other such that the annular elements maximize the volume occupied by the guide element in the intravascular access device. In some examples, the geometry of the annular elements may complement one another such that they promote optimized steerability or function of the guide element. In some examples a guide element may have two or more annular elements that can be generally cylindrical (e.g., circular cross-sections). In some examples, the dimensions of the guide element and/or each annular element may consider the function or deployment route through the intravascular access device. For example, an annular element may have a diameter between 0.0055″ and 0.0075″. In some examples, each annular element may have a width between 0.0060″ and 0.0070″. In some examples, the width of an annular element may be 0.0065″. In some examples, the width of an annular element may be relative to the width of one or more other annular elements of the same guide element. In some examples, the width of an annular element may be 0.00075″, 0.001″, 0.00125″, 0.0015″, 0.00175″, 0.002″, 0.00225″, 0.0025″, 0.00275″, 0.003″, 0.00325″, 0.0035″, 0.00375″, 0.004″, 0.00425″, 0.0045″, 0.00475″, 0.005″, 0.00525″, 0.0055″, 0.00575″, 0.006″, 0.00625″, 0.0065″, 0.00675″, 0.007″, 0.00725″, 0.0075″, 0.00775″, 0.008″, 0.00825″, 0.0085″, 0.00875″, 0.009″, 0.00925″, 0.0095″, 0.00975″, 0.01″, 0.01025″, 0.0105″, 0.01075″, 0.011″, 0.01125″, 0.0115″, 0.01175″, 0.012″, 0.01225″, 0.0125″, 0.01275″, 0.013″, 0.01325″, 0.0135″, 0.01375″, 0.014″, 0.01425″, 0.0145″, 0.01475″, 0.015″, 0.01525″, 0.0155″, 0.01575″, 0.016″, 0.01625″, 0.0165″, 0.01675″, 0.017″, 0.01725″, 0.0175″, 0.01775″, 0.018″, 0.01825″, 0.0185″, 0.01875″, 0.019″, 0.01925″, 0.0195″, 0.01975″, 0.02″, 0.02025″, 0.0205″, 0.02075″, 0.021″, 0.02125″, 0.0215″, 0.02175″, 0.022″, 0.02225″, 0.0225″, 0.02275″, 0.023″, 0.02325″, 0.0235″, 0.02375″, 0.024″, 0.02425″, 0.0245″, 0.02475″, 0.025″, 0.02525″, or more. In some examples, each annular element may have a different width. In some examples, more than one annular element may have the same width.

In some examples, the annular elements may be contacting or substantially adjacent to one another. For example, the annular elements may be generally parallel with one another along a segment of the guide element. In some examples, the annular elements may be separated from one another by a gap (e.g., two annular elements may be separated by a gap of 0.004″). In some examples the guide element may have a comprehensive cross section width (e.g., two annular elements, a gap between them may be 0.017″).

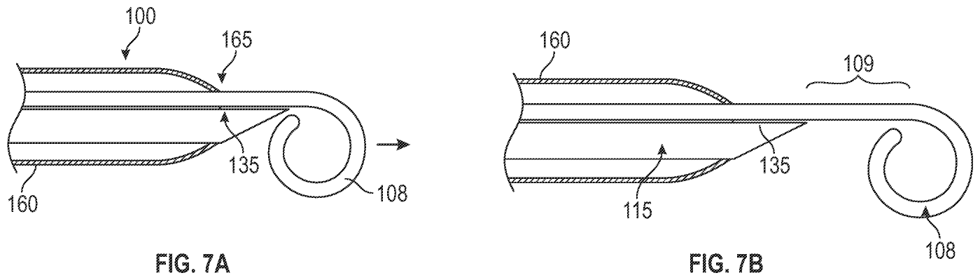

A guide element may be used with an intravascular access device having a catheter 160 with a catheter distal tip door 165 configured to facilitate a transition of the guide element 105 from a ready configuration to an advanced or in-use configuration. FIGS. 7A and 7B show examples of longitudinal cross-sectional views that illustrates the distal end of the intravascular access device 100 (e.g., catheter of an intravascular access device) and the guide element having the curled distal end 108 curling over the needle distal tip. The guide element is extending outward from the working space 130 between the catheter 160 and the needle exterior surface. The needle flat surface 135 extends along the needle body to the needle distal tip and a catheter distal tip 165 configured to accommodate the transition of the guide element distally from inside the working space into the vessel is shown in an open position biased opened by the advanced guide element loop 108 passing therethrough.

In some examples, the distal end, portion, segment, etc. of the catheter may be configured to expand or have increase elasticity or flexibility to allow for the guide element to be extended distally from inside the catheter to inside the vessel. For example, a region adjacent to the distal tip of the catheter may comprise a material having a durometer rating less than a proximal portion or segment of the catheter. In this way, the catheter may comprise a distal opening configured to contact the exterior surface of the needle when the catheter is initially inserted into the vessel. Then, after the catheter has been inserted, the guide element may be advanced distally from inside the working space and the distal perimeter of the catheter can expand or flex allowing the guide element to pass through the catheter distal perimeter and into the vessel. In some examples, the catheter distal perimeter may be configured to contract around the needle exterior surface. In some examples, the catheter distal perimeter may be configured to contract around the needle exterior surface and the guide element exterior surface when the guide element is advanced into the vessel. An extended configuration is shown in FIG. 7B where the guide element has been extended to length 109 beyond the needle tip, maintaining the curled distal end 108. The catheter distal door is a slit or other opening to allow the guide element to pass therethrough while maintaining a maximum amount of continuous contact around the needle perimeter. From these cross sectional views, the guide element may be extending through a slit in the distal end of the catheter, through an elastic perimeter of the distal end of the catheter, or a combination thereof.

In some examples, the catheter may comprise one or more materials such as silicone rubber, nylon, polyurethane, polyethylene terephthalate (PET), latex, polyimides, thermoplastic elastomers, etc. Any of the catheters described herein may comprise one regions (e.g., segments, portions, areas, features, etc. with increase flexibility relative to other segments, portions, regions, areas, features, etc. For example, a durometer rating of the catheter or catheter region may be measured according to a Shore 00, Shore A, or Shore D scale. In some examples, the durometer rating of the catheter or catheter region may be 0, 10, 20, 30, 40, 50, 60, 70, 80, 90, 100 or more or any value therebetween according to the Shore 00, Shore A, or Shore D scale. For example, the elongate body of a catheter may comprise a higher durometer rating compared to the distal end, distal perimeter, deployment slit(s), etc. or a combination thereof. For example, the elongate body of a catheter may be Shore A 40, while the distal region (e.g., distal perimeter) of the catheter may be Shore A 20.

In some examples, the catheter may comprise one or more materials such as silicone rubber, nylon, polyurethane, polyethylene terephthalate (PET), latex, polyimides, thermoplastic elastomers, etc. Any of the catheters described herein may comprise one regions (e.g., segments, portions, areas, features, etc. with increase elasticity relative to other segments, portions, regions, areas, features, etc. For example, the catheter distal end, distal region, distal perimeter may have an increased elasticity configured to expand the distal opening and allow the guide element to be advanced distally from the working space.

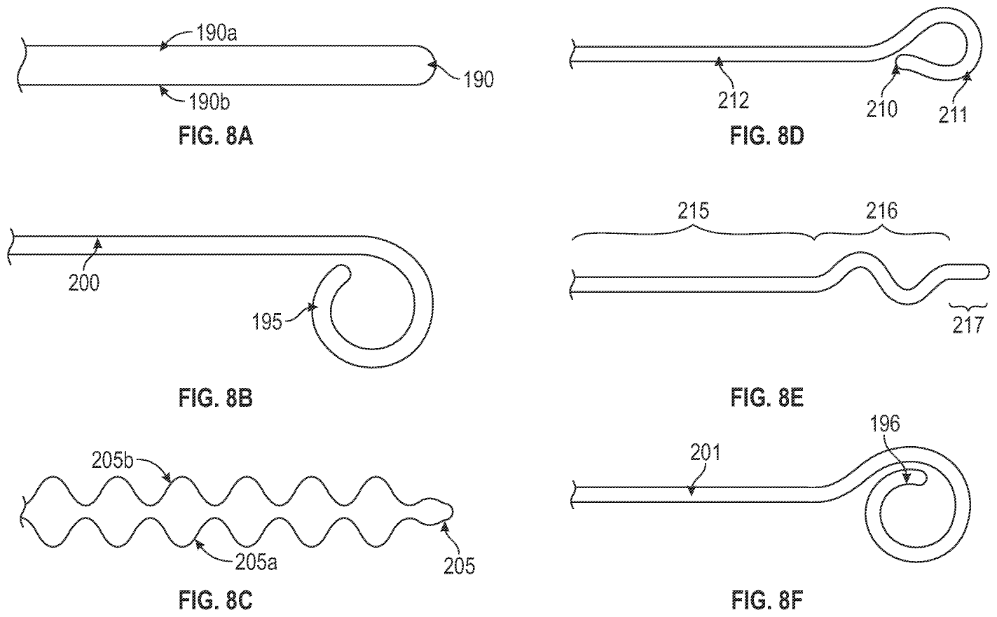

In some examples, the guide element may have shape memory in one or more of the annular elements. FIGS. 8A to 8E illustrate examples of shape memory of one or more of the annular element of the guide element. The shape of the guide element along a segment of its body or near the distal end may provide a benefit in the function and operation of the guide element during a procedure. In some examples, the guide element may initially be retained in a linear or substantially linear shape in a ready or retracted configuration. As the guide element is advanced distally from the intravascular access device, the shape memory may present in any configuration predetermined and imparted into the guide element and or guide element components.

Referring to FIG. 8A, and in some examples, guide elements described herein may comprise an atraumatic tip at the distal end of an elongate body. Here, the elongate body comprises two members 190a and 190b. In some examples, members 190a and 190b may be advanced simultaneously or at a different rate relative to one another. Where they are advanced at different rates, (e.g., member 190a advanced faster than member 190b), the atraumatic tip 190 may be a different segment of the total length of the guide element such that the characteristics of the atraumatic tip are maintained through the length of the guide element forming the same. In FIG. 8B, this guide element example comprises a loop 195 as an atraumatic distal end with a segment of the guide element curling after it has been advanced from the catheter. For example, the elongate body 200 may be coaxial to the distal tip of the guide element when stowed in the catheter, then the distal segment 195 may curl when the guide element is advanced beyond the needle tip. In FIG. 8C, an example of variations in the shape of the guide element elongate body is shown. Here, the atraumatic tip 205 is locatable at a distal end of two non-linear members 205a and 205b comprising the elongate body. Accordingly, in some examples, guide elements described herein may have characteristics and attributes to improve navigation through the vessel, or provide additional support and function such as expanding the vessel or addressing obstructions when advanced into the vessel. FIG. 8D, shows another example of a guide element with a hook end 211 where the distal end 210 does not curl entirely around at the distal region of the guide element. In some examples, as illustrated in FIG. 8E, a guide element may be considered based on different segments or regions of the guide element. For example, a proximal region 215 may comprise materials having different stiffness or compositions to provide column support and strength when advancing the guide element. Region 215 may comprise a shape memory or material composition configured to facilitate changes in the form and function of the guide element. Finally, the distal end 217 may be configured to provide an atraumatic tip or other attribute or improved deployment and navigation. Yet another example of a guide element is illustrated in FIG. 8F with a distal curl 196 at the end of an elongate body 201. Here, the curl may be more than 360 degrees of curl. In some examples, a guide element may comprise a distal end with multiple curls at the distal end. Accordingly, the distal end may be configured to provide increase flexibility such as a spring when navigating tortuous vessels.

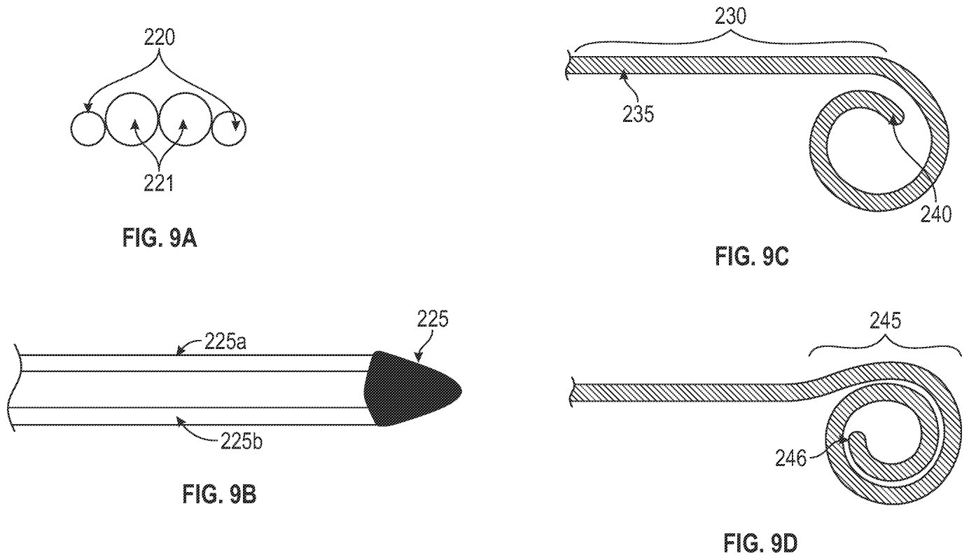

FIGS. 9A to 9D show additional examples of guide elements, as described herein. In FIG. 9A, an example of a cross section of a guide element segment shows multiple annular elements (e.g., 220 and 221) arranged in a generally linear fashion relative to one another as they may be on top of the needle flat surface in the working space before, during, or after distal advancement. Their cross-sectional geometry is generally circular but could be any geometry that may facilitate control and operation of the guide element and/or the guide element distal end or tip during a procedure. With multiple annular elements (e.g., multiple annular elements with the same or different characteristics), there may be additional capabilities to control and operate the guide element. For example, the two central annular elements 221 may allow for vertical control or steering of the guide element within the vessel or may allow for increased structural integrity when advancing the guide element into the vasculature. The lateral annular elements 220 may provide for lateral control. For example, advancing the guide element distally at first may be done by advancing any one or all the annular elements, then an operator may steer the distal tip to one side or another by retracting or advancing the lateral annular elements 220. FIG. 9B illustrates another example of variation for guide elements described herein having a molded or affixed distal tip 515. In the example shown in FIG. 9B, there are multiple annular elements 225a and 225b. However, a molded or affixed distal dip on a guide element may be locatable at the distal end of a single elongate body (e.g., annular element) or a plurality of annular elements (e.g., 225a and 225b). As illustrated in FIGS. 9C and 9D guide element distal tip 204 and 246 may be capable of up to a 180 degree turn based on the manipulation of one or more annular elements. For example, one or more annular elements may actuate the transition from a linear guide element configuration to a curled atraumatic distal tip. Of note in FIG. 9C and 9D, the guide element may be comprised of a braided material. For example, FIG. 9C illustrates a length 230 of elongate body 235 that may be a braided or woven structure. In some examples, the braided or woven structure of the guide element may be coated or covered (e.g., a biocompatible polymer, metal, etc.). In some examples, a guide element described herein may have a curled or curved atraumatic distal tip that can change from a first position (e.g., a linear shape in a ready configuration) to a second position whereby the distal tip curls, curves, loops, or otherwise changes from a linear shape at or near the distal tip when the guide element is advanced beyond the distal end of the intravascular access device. For example, FIG. 9D illustrates multiple loops or curls of the distal region 245 of the guide element. In some examples, the distal end of the guide element may be curled, looped, or otherwise in a non-linear configuration inside of the intravascular access device before it has been advanced (e.g., in a ready to use configuration).

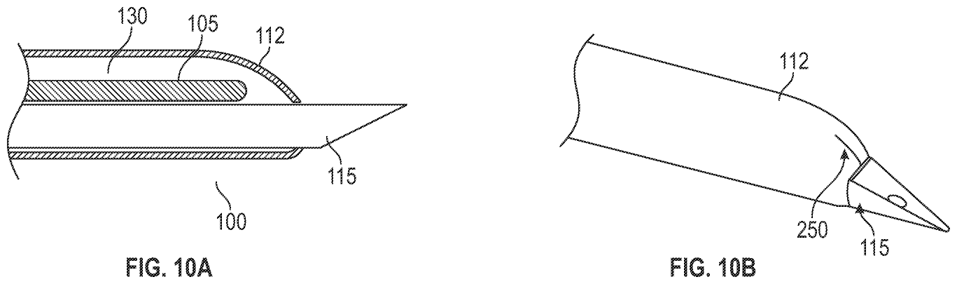

FIG. 10A shows an example of a guide element and intravascular access device as described herein. A distal end of an intravascular access device 100 is illustrated as a longitudinal cross-sectional view highlights a retracted or ready configuration of the guide element 105 within the working space 130 between the needle 115 (e.g., the needle flat surface) and the interior surface of the catheter 112. The working space may be at any position longitudinally along the length of the needle and catheter at the distal end of the intravascular access device relative to the arrangement and orientation of the needle flat surface. For example, FIG. 10A illustrates the needle 115 having the distal tissue penetrating tip aligned at a position such that the guide element would advance distally along the longest length of the needle 115 until it has advanced beyond the tissue penetrating tip. The flat surface may longitudinally along the needle body at any position (e.g., radial or clock position relative to the needle axis). In some examples, there may be more than one working space whereby the needle of the intravascular access device may have more than one flat surface (e.g., a square or other polygon geometry) creating a working space with each flat surface and the interior surface of the catheter.

In FIG. 10B, an intravascular access device, as described herein is illustrated having a catheter with a distal end comprising a deployment slit 250 (e.g., catheter distal tip door). The deployment slit 250 may be configured to facilitate the advancement or other operation of the guide element from within the working space as it would be advanced distally from the intravascular access device. For example, the guide element may be advanced distally against the catheter distal end with sufficient force or pressure to open or separate the deployment slit 250 and then extend or be capable of being advanced beyond the distal end of the catheter. In some examples, the catheter distal slit may be a single slit or a plurality of slits. In some examples, the arrangement of the deployment slit 250 may be associated with the location or arrangement of the intravascular access device working spaces. For example, the deployment slit 250 may be aligned with a distal terminus of a working space. In some examples, a plurality of deployment slits may be arranged relative to one another such that they form a flap or catheter distal tip door (e.g., as described above). In some examples, the distal end of the catheter is elastic and may contour the exterior surface of the needle in a ready configuration (e.g., as the needle and catheter are inserted into a vessel). In some examples, when the guide element is advanced through the working space, the catheter distal end may expand against an elastic contraction force against the needle flat surface to create a seal around the advanced guide element and the needle to prevent unnecessary flow of fluid (e.g., blood from within the vessel through the catheter working space.

In some examples, deployment slits may be a perforated line or lines arranged around the distal end of the catheter to provide or facilitate passage of the guide element distal tip transitioning from a stowed configuration to a deployed configuration. In some examples, the deployment slit or slits may indicate a line or region of increase flexibility such that the catheter exterior surface remains continuous, yet the deployment slits can be configured to expand to allow the guide element to transition from the stowed configuration to the deployed configuration.

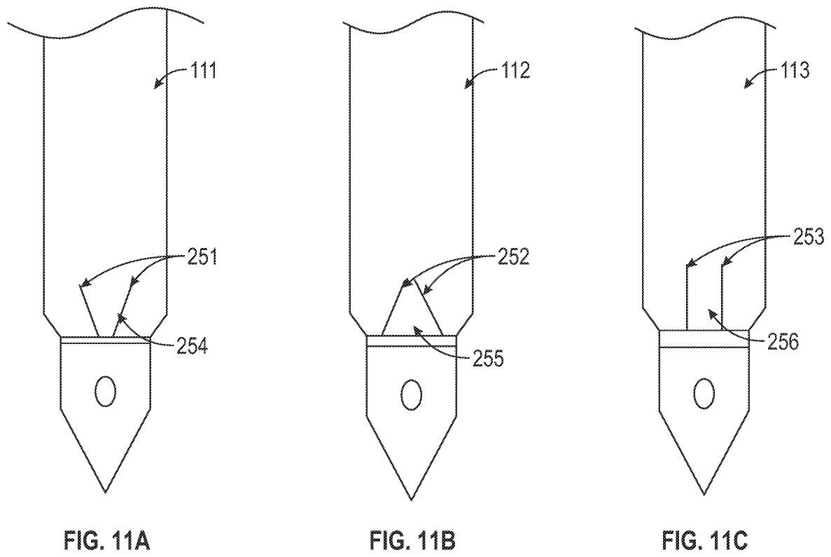

The deployment slit arrangement may include the orientation of the slit from the distal end of the catheter. For example, FIGS. 11A to 11C illustrate deployment slits at various angles or arrangements at or near the distal end of the catheter to facilitate the operation of the guide element or another tool being passed through or advanced outward from within the intravascular access device (e.g., the working space). In some examples, the deployment slits may also facilitate one or more annular elements of the guide element. For example, as seen in FIG. 4, where the guide element may extend within or through the needle aperture and continue through the working space, the deployment slit may facilitate the transition of the guide element (e.g., one or more of the annular elements) from the needle aperture and into or through the working space.

Referring to FIG. 11A, deployment slits 251 are angles and configured to provide a minimized opening in the perimeter of the distal end of the catheter. In some examples, a passage, flap, door, etc. may be created when the guide element is advanced against deployment slits 251. Similarly in FIG. 11B, deployment slits 252 are angled such that the distal end of the flap or opening is larger around the perimeter of the catheter distal end. In some examples, the geometry of a deployment region or area of the catheter distal end may be configured to accommodate a tool or guide element distal end having a complementary geometry. For example, a guide element or tool advancing distally from within the catheter that has a larger distal tip may benefit from a catheter such as the one illustrated in FIG. 11B with a larger opening or elastic region at the distal perimeter of the catheter. Finally, FIG. 11C illustrates yet another example of a guide element deployment area on the distal of the catheter 113 with parallel deployment slits 253 providing a region or flap configured to facilitate the distal advancement of the guide element distal tip from inside the catheter.

In some examples, any of the catheters described herein may comprise a continuous distal end whereby a segment, region, perimeter, or area of the catheter distal end may be configured to allow distal advancement of the guide element from within the catheter without disrupting the continuous exterior surface of the catheter. For example, the flaps or doors provided by deployment slits described herein may represent an area of decreased structural integrity or increase elasticity allowing the guide elements to be deployed from within the catheter while the catheter distal end maintains substantial contact with the exterior surface of the needle and guide element extending therethrough. For example, in any of the catheters described herein, the distal end may be configured to facilitate deployment of a guide element without disrupting the structure of the catheter itself.

In any example, the deployment region (e.g., deployment slits, door, flap, elastic region, elastic perimeter, distal region with lower durometer rating, etc.) may be configured to facilitate the introduction of a tool or other interventional device deployed from within the catheter through the working space between the exterior surface of the needle (e.g., needle flat surface) and the interior surface of the catheter). For example, a cutting device, suction device, imaging device, light, balloon, etc. may be deployable from within the catheter.

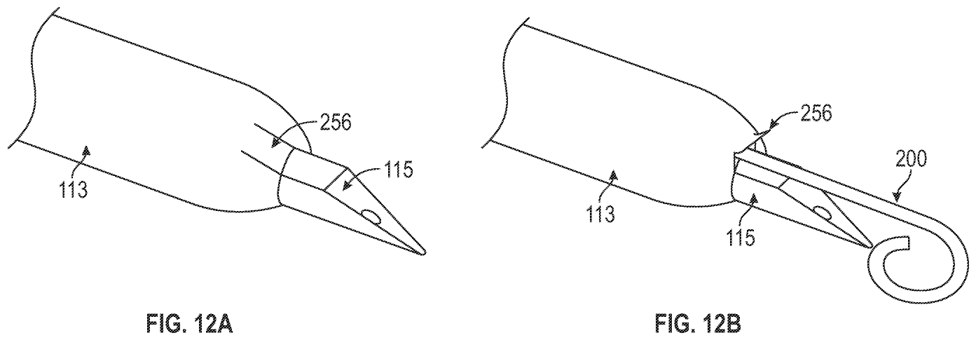

FIGS. 12A and 12B illustrate examples of transitions for the guide element including the deployment slit or catheter distal door created by more than one deployment slit. For example, FIG. 12B shows the guide element 200 extending outward from the deployment opening 256 with the curled tip of the guide element advancing forward and distally beyond the needle 115. Referring to FIG. 12A, the guide element is in the stowed or ready-to-use configuration before being deployed from inside the catheter 113 and working space therein. In FIG. 12B, the guide element 200 has been advanced distally through opening 256 and continued beyond the needle 115.

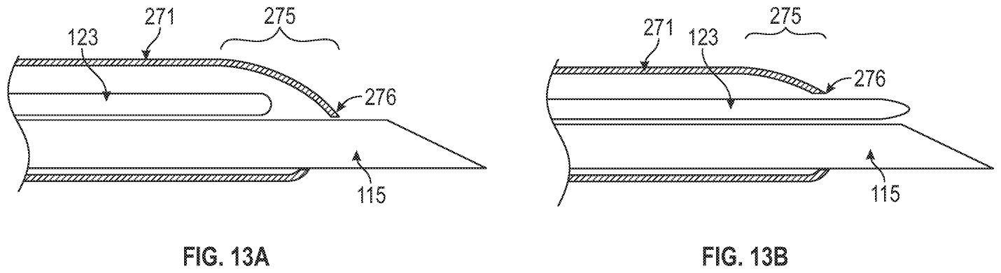

Similarly, the longitudinal cross-sectional views of FIGS. 13A and 13B illustrate an example of an elastic distal end 275 or segment of a catheter 271. The distal end of the catheter 276 is contacting the exterior surface of the needle 115 in FIG. 13A such that he guide element 123 is retained inside the working space and in the stowed configuration. Then, in FIG. 13B, the guide element 123 has been deployed against the elastic perimeter 276 such that the catheter perimeter is now contacting the exterior surface of the needle and the exterior surface of the guide element extending therethrough.

In some examples, the distal tip or region of the catheter having a deployment feature (e.g., catheter door/deployment slit) to facilitate transition, advancement, or other function of the intravascular device (e.g., guide element). In some examples, the deployment feature is the deployment slit or configuration of slits that may provide a door, flap, or otherwise selectively openable catheter distal lumen allowing a guide element and/or needle to extend therethrough. In some examples, the deployment feature may be a region of expandable (e.g., elastic or compressive) distal lumen of the catheter that may be biased around the outer perimeter of the intravascular access device (e.g., needle) that may seal or enclose the working space until a guide element is advanced through the deployment feature. For example, a guide element may be locatable within the working space and advanced against compression of the distal lumen (e.g., deployment feature) of the catheter to sufficiently expand an opening allowing the guide element to be advanced therefrom. In some examples, the distal lumen of the catheter (e.g., deployment feature) may confirm to the outer perimeter of the intravascular access device including a perimeter of the guide element extending therethrough.

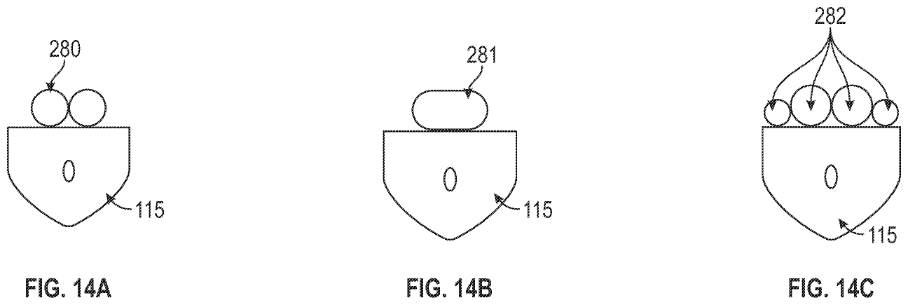

In some examples, the catheter distal end or distal tip may have a confirming segment to conform to the distal tip of the guide element. The guide element have a formed or connected distal tip with a geometry that is complemented by the conforming segment of the catheter distal tip. In such a configuration, the guide element distal tip may be in a ready to use configuration when the distal tip is seated within the catheter conforming distal segment. FIGS. 14A, 14B and 14C provide additional illustrative examples of a guide element annular element configuration with consideration given to the needle 115. The number of annular elements, the arrangement of annular elements, and the size of annular elements may be based on or associated with the dimensions of the working space (e.g., the length or surface of the needle flat surface and/or the volume of the working space created by the needle flat surface and the interior of the catheter. For example, FIG. 14A illustrate two annular elements 280 while 14B illustrates a single annular element 281 with an oblong cross-sectional geometry and yet another example of multiple annular elements 282 is illustrated by FIG. 14C. FIG. 14B illustrates further detail of an example of the coupling or attachment of the guide element with the guide element distal tip. Shown here is an example of the guide element affixed, coupled, or otherwise engaged to the formed distal tip. The distal tip of the guide element may be formed into an atraumatic geometry to increase safety and improve maneuverability within a vessel.

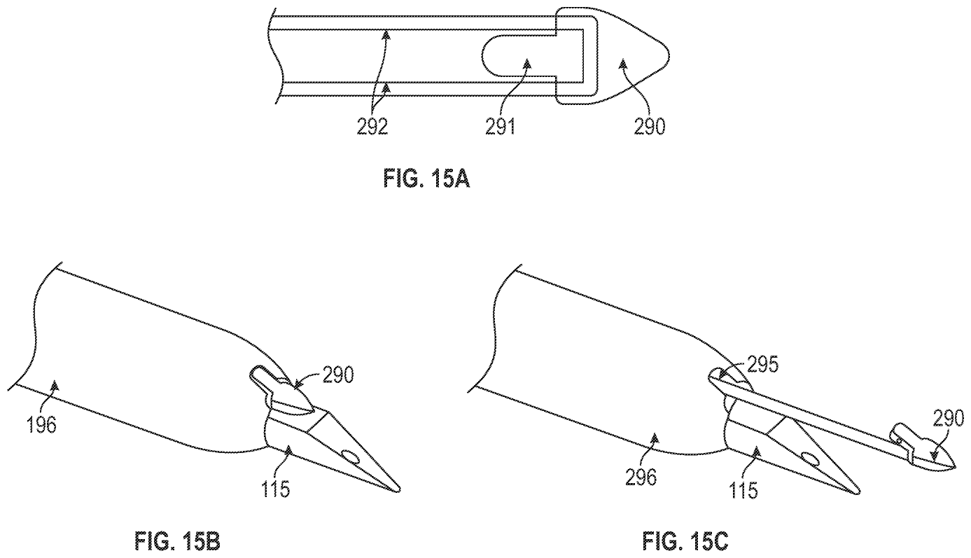

Yet another example of guide element configurations are illustrated in FIG. 15A to 15C. In FIG. 15A, the distal segment of a guide element is shown with a molded or affixed distal tip 290 at the distal end of an elongate body 292 comprising two annular elements. On the proximal side of the distal tip 290 is an engagement feature 291 configured to seat within a corresponding feature or element of the catheter distal tip. Referring to FIG. 15B, the guide element distal tip 290 is seen in the ready position and seated in the corresponding features or notch of the catheter distal end. Here, the guide element distal tip 290 is also shown in a configuration to provide a smooth transition at the catheter distal end. For example, the guide element distal tip 290 may be configured to seat in or otherwise engage the distal end of the catheter in such a manner as to complete the distal circumference or perimeter of the catheter distal end. In this way, when the catheter 196 is inserted into a vessel, there is a minimal amount of dilation required for insertion. Additionally, in FIG. 15B the guide element distal tip 290 is seated in the conforming segment of the catheter distal tip and then as the guide element is advanced, the distal tip moves distally from the conforming segment of the catheter tip beyond the needle Then, in FIG. 15C, the guide element distal tip 290 has been advanced from the catheter feature 295 and may be advanced into the vessel.

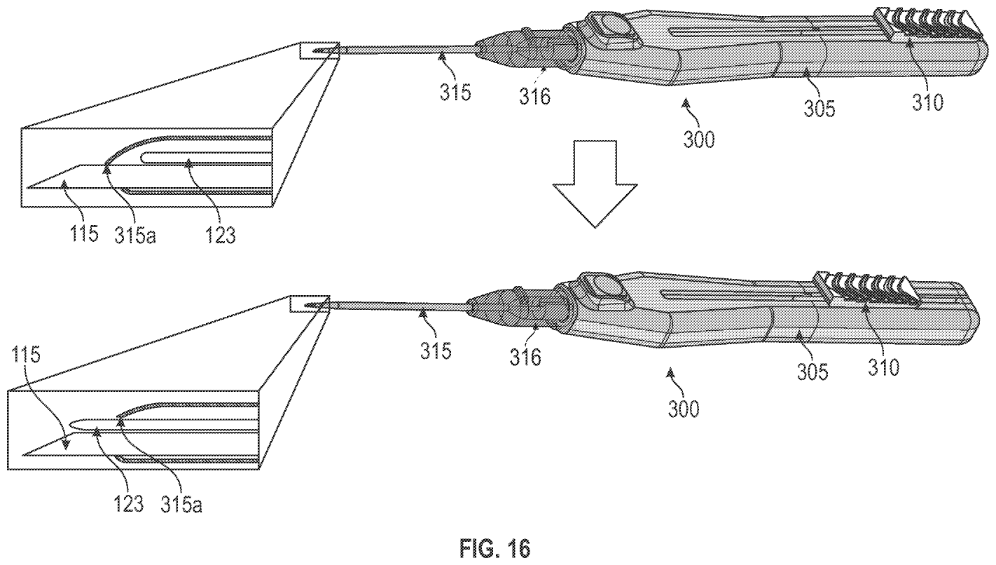

FIG. 16 illustrates an example of a guide element in use as it is deployed from within the working space by an intravascular access device 300. The intravascular access device 300 has a handle 305 and a slider 310 in operable communication with the guide element 123. The catheter 315 extends from the catheter hub 316 coupled to the distal end of the intravascular access device 300. Expanded views of the catheter distal end show the catheter 123 in a stowed position within the working space outside of the needle 115 and inside of the catheter 315. In this example, the catheter distal end 315a may be an example of a region having increased elasticity, decreased durometer rating, or otherwise configured to contract around the needle exterior. That is, until the slider 310 is advanced, thereby advancing the guide element 123 distally along the exterior surface of the needle 115 and through the catheter distal end 315a. As the slider 310 and guide element 123 are advanced, the guide element distal end contacts the interior of the catheter distal end 315 to expand the catheter distal end 315a or otherwise deploy from the catheter 315. In some examples, the catheter distal end 315a may then maintain contact with the guide element 123.

In some examples, the distal end of the guide element may be configured as a spring. For example, a curved or looping at the distal segment of the guide element may provide increased structural integrity compared to a distal segment being partially loop, whereby the distal segment may be more compressible about the curve. In some examples, the curve or loop may provide for a spring capable of absorbing a force or contact between the guide element distal end and the patient's anatomy.

In some examples, a guide element may have multiple annular element segments. In some examples, the annular element segments define a length of the guide element from the distal segment of the guide element to a proximal portion of the guide element. For example, there may be four annular element segments. In some examples, there may be one annular element segment, two, three, four . . . to an amount of annular element segments sufficient to promote optimized function and operation of the guide element and the associated intravascular access device. In some examples, the number of annular element segments may be related to the maneuverability of the guide element as it advances. For example, one or more of the annular element segments may be selectively displaced based on a desired travel or appreciation of an obstacle within the vasculature.

In some examples, the distal end of the catheter may have a separation in a continuous circumference that is selectively opened by advancing the guide element through the distal end of the catheter. The orientation of the separation or slit may be based on the configuration of the guide element distal geometry; the deployment function of the guide element; the size of the guide element; the vessel or vessel location where the intravascular access device is deployed; the distal tip of the guide element; or more.

Some examples of materials comprising the guide element, or a guide element segment may include nitinol, PEEK, or another material having sufficient rigidity and stiffness to support advancement through vasculature. Additionally, the material may have memory wherein a shape may be imparted into the material through molding or other forming means such that the shape is restricted in a retracted state and the shape is re-established after the shaped portion is advanced out of the IV device/system.

In some examples, the guide element and/or one or more of the guide elements components may comprise one or more alloys. In some examples, the alloy composition of the guide element may be different at different areas of segments along the length or at various segments of the guide element. For example, the distal end may comprise one or more allows configured to adapt, adjust, or otherwise change a shape or orientation of the alloy segment. For example, the distal tip may be a first alloy, a segment adjacent to the distal tip may be a second alloy, and a segment adjacent thereto may be a third alloy. The second alloy may have properties that are different than the first and third alloys causing the second alloy segment to change, confirm, or otherwise react to different environmental factors causing a predetermined or desirable change in the configurations and orientation of the guide element distal end. In some examples, an alloy segment may contract at a faster rate in the presence of lower temperatures compared to other allows resulting in predetermined curves or bends at said segment. In some examples, the guide element may have more than one annular segment and each annular segment may comprise a different alloy and/or be comprised of materials having different attributes such as stiffness, malleability, hardness, conductivity, etc. For example, one annular element may comprise a material of sufficient hardness to be advanced through an occlusion without causing a bend or kink in the guide element as it is advanced therethrough, while a second annular element may have less stiffness that may bend if the guide element were advanced only using the softer annular element. In such a configuration, the soft sided annular element may act as a test element when advancing the guide element through vasculature and allow for testing of the amount of force necessary to advance the guide element through an occlusion.

Guide elements (e.g., guide wires and associated structures) described herein may be used during a medical procedure (e.g., intravascular access) and/or in association with an intravascular access device. Intravascular access devices (e.g., any element of an intravascular access device) described herein may be used and/or associated with a medical procedure.

There may be one or more variations, alternatives, constructions, compositions, and/or components described herein that can be used to modify an element, component, device, system, process, etc. of a guide element, intravascular access device and/or an associated structure or process. Accordingly, any variation, description, example, element, component, process, method, method step, etc. described herein can be used as a modification, variation, and/or alternative to any element, device, system, composition, example, component, process, method, method step, etc. described in PCT application number PCT/US23/65556, filed on Apr. 7, 2023, entitled “INTRAVASCULAR CATHETER WITH INTEGRATED GUIDE STRUCTURE”; and/or PCT application number PCT/US21/54046, filed on Oct. 7, 2021, entitled “INTRAVASCULAR CATHETER WITH INTEGRATED GUIDE STRUCTURE” the entireties of which are incorporated herein.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled”to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A guide element for an intravascular access device comprising:
a catheter body having a distal end, a proximal end and a lumen extending therethrough;
an intravascular access device having a needle inside of the catheter lumen and a working space between an exterior surface of the needle and an interior surface of the catheter, the working space configured to receive one or more tools therethrough; and
a guide element within the working space wherein a portion of the catheter body adjacent to the guide element is displaced in response to the distal movement of the guide element along the needle within the working space.

2. The guide element of claim 1, wherein the portion of the catheter body is displaced by moving a flap formed on the catheter body in response to the guide element.

3. The guide element of claim 2, wherein the portion of the catheter body is displaced by the guide element has a different durometer than the rest of the catheter body.

4. The guide element of claim 3, wherein the portion of the catheter body having a different durometer that may be displaced by movement of the guide element is an annular portion at a distal most portion of the catheter body.

5. The guide element of claim 3, wherein the portion of the catheter body having a different durometer that may be displaced by movement of the guide element is adjacent to the working space or in a semicircular portion that corresponds to a flat portion of the needle.

6. The guide element of claim 1, wherein the catheter body has a first portion formed from a material of a first durometer and a second portion formed of a material of a second durometer wherein the first portion is adjacent to the working space and the first durometer is selected such that in use as a distal end of the guide element is advanced against the first portion, the first portion deflects or deforms in response.

7. A guide element for an intravascular access device comprising:
an intravascular access device having a needle inside of a catheter lumen and a working space between an exterior surface of the needle and an interior surface of the catheter, the working space configured to receive one or more tools therethrough; and
a guide element having a distal end and a proximal end separated by one or more annular elements, the guide element configured to be distally advanced from the intravascular access device inside of a blood vessel, wherein the one or more annular elements is configured to control at least a distal end of the guide element.

8. The guide element of claim 7, further comprising at least two annular elements, wherein a first annular element is configured to control a direction of travel of the guide element distal end.

9. The guide element of claim 7, wherein the distal end of the guide element is an atraumatic tip.

10. The guide element of claim 7, wherein the distal end of the guide element is a formed distal tip.

11. The guide element of claim 7, wherein the guide element is comprised of a plurality of segments.

12. The guide element of claim 7, further comprising at least one annular element.

13. The guide element of claim 7, wherein the needle has at least one flat, convex, or concave surface, and wherein the working space is defined by an interior arc of the catheter interior surface and the needle surface.

14. The guide element of claim 7, wherein the catheter distal tip comprises one or more deployment slits aligned with the working space.

15. The guide element of claim 7, wherein the catheter distal tip comprises one or more conforming segments, wherein a distal tip of the guide element is configured to engage the one or more confirming segments.

16. An intravascular access device, comprising:
a handle having a proximal end and a distal end, a slot extending from the proximal end to the distal end;
a catheter having a proximal catheter hub and a distal catheter lumen, the proximal catheter hub releasably engaged to the distal end of the handle;
an access needle extending proximally from within a needle carrier through the catheter lumen, the access needle having at least one flat surface extending longitudinally along a perimeter of the access needle, and a tissue-penetrating tip extending distally beyond the catheter lumen;
a working space within the catheter along a length of the access needle having a nonuniform circumferential cross-sectional geometry, wherein the working space is configured to facilitate passage of one or more tool therethrough from a proximal end of the intravascular access device distally into a vessel; and
a slide extending through the slot, the slide in communication with a proximal end of the guide element so that distal advancement of the slide advances a distal tip portion of the guide element from a position within a guide element cutout distally along the surface of the access needle.

17. The intravascular access device of claim 16, wherein at least one flat surface of the access needle extends longitudinally down an exterior surface of the access needle.

18. The intravascular access device of claim 16, wherein the at least one tool comprises a guide element.

19. The intravascular access device of claim 16, wherein the working space is defined by a needle flat surface and an interior surface of the catheter, wherein the working space is selectively accessible from a proximal end of the intravascular access device.

20. The intravascular access device of claim 16, wherein the working space is configured to selectively open at the catheter distal end and wherein the working space is configured to guide a tool or tool segment sliding therethrough.

21.-28. (canceled)
